(19) United States
(12) Patent Application Publication
(10) Pub. No.: US 2009/0204064 A1
(43) Pub. Date: Aug. 13, 2009

(54) INSTRUMENT AND METHOD FOR THE ENDOSCOPICALLY CONTROLLED SHORTENING AND/OR FRAGMENTATION OF STENTS LOCATED IN HOLLOW ORGANS

(76) Inventors: Günter Farin, Tübingen (DE); Karl Ernst Grund, Tübingen (DE)

Correspondence Address: DICKSTEIN SHAPIRO LLP, 1825 EYE STREET NW, Washington, DC, 20006-5403, US

(21) Appl. No.: 12/093,247

(22) PCT Filed: Nov. 9, 2006
(86) PCT No.: PCT/EP06/10770

§ 371 Date: Nov. 14, 2008

(30) Foreign Application Priority Data
Nov. 10, 2005 (DE) 10 2005 053 764.2

(51) Int. Cl. A61B 18/18; A61M 37/00

(52) U.S. Cl. 604/26; 606/108

(57) ABSTRACT

An instrument and a method are proposed for endoscopically controlled shortening and/or fragmentation of stents made from electrically conductive material situated in the gastrointestinal tract, in the tracheobronchial system or in other hollow organs. The instrument comprises an electrode device with an electrode for introducing a high-frequency current into at least one wire of the stent and/or to form electric arcs between the electrode and the at least one wire. Use of the instrument and the method results in little or reduced damage being caused by current and/or heat in tissues directly adjacent to the application site and in tissues remote therefrom.

The invention relates to an instrument for endoscopically controlled shortening and/or fragmentation of stents situated in the gastrointestinal tract, in the tracheobronchial system or in other hollow organs.

Stents are essentially elastic tubes whose walls are made from special metal wires in meshes of various sizes, for example, by braiding or knitting.

Stents are used to an ever increasing extent for palliative treatment of stenosing tumours or scar tissue, for covering or closing anastomotic insufficiencies, fistulae and the like, for bridging necrosis cavities and the like in the gastrointestinal tract and the tracheobronchial system. Stents are used preferentially in these instances. When correctly implanted, stents lie closely against the respective organ wall with a greater or smaller elastic force in order to ensure the passage of solid, liquid and/or gaseous substances through the hollow organ in question.

If a stent is incorrectly implanted, damaged during or following implantation, or is insufficient in some other way, it may be necessary to shorten it and/or to remove it entirely. This may be problematic, since an advantage of stents, namely their good, secure frictional fixation on the organ wall, hinders their removal. Particularly problematic is the removal of stents when they lie in curves of hollow organs and/or are deformed, or if turnout or other tissue has grown inwards from outside through the mesh of the stent. Previously no special method or instrument has been available for shortening and/or complete removal of stents in or from the gastrointestinal tract, the tracheobronchial system or in/from another region. Conventionally, for shortening, given the lack of better methods or instruments, thermal methods were used wherein the metal wires of stents were heated to their melting point at sites suitable for shortening or fragmentation, and thereby parted. For this purpose, endoscopically usable lasers, in particular Nd:YAG lasers or argon plasma are used. However, the conventionally available, endoscopically usable Nd:YAG lasers and argon plasma applicators are designed for thermal haemostasis and/or thermal devitalisation, coagulation or desiccation, but not for melting metal wires. Both methods can cause unintended thermal damage in the tissues immediately adjacent to and/or remote from the application site. Use of Nd:YAG lasers is also expensive and involves observing extensive safety regulations.

It is an object of the invention to provide endoscopically usable instruments and a method for shortening and/or fragmentation of stents situated in the gastrointestinal tract, in the tracheobronchial system or other hollow organs, wherein damage to tissues immediately adjacent to and/or remote from the application site with the instrument and the method are avoided as far as possible.

This object is achieved with an instrument according to claim 1 and a method according to claim 40.

From the standpoint of the device, the object is achieved, in particular, with an instrument for endoscopically controlled shortening and/or fragmentation of stents made from electrically conducting material and situated in the gastrointestinal tract, the tracheobronchial system or in other hollow organs, which instrument comprises an electrode device with an electrode for introducing an HF current into at least one wire of the stent and/or for forming electric arcs between the electrode and the at least one wire. A protective device is also provided which is configured and mechanically connected to the electrode device such that the wire can be thereby separated and/or distanced from the tissue of the gastrointestinal tract, tracheobronchial system or other hollow organ on which it lies or by which it is surrounded, during introduction of the HF current and/or during the formation of electric arcs.

An essential point of the invention lies therein that, by means of a single instrument and a suitable corresponding method, individual wires (or small groups of wires) of the stent are separated from the tissue adjacent to them, so that when the HF current is introduced and/or the arc is formed to heat the wire, cooling by means of the tissue can no longer take place and damage to the tissue is at least minimised.

Arranged at a distal end of this instrument is an electrode device which comprises the active electrode and which either, for direct heating of stent wires, touches these stent wires or, for indirect heating of stent wires, is spaced apart from these stent wires in order to generate the electric arcs necessary for indirect heating of said stent wires. Thus, for electrical heating of a metal stent wire, either an electric current which heats it directly, that is from inside, must be passed through it, or an electric arc which additionally, or overwhelmingly, heats the stent wire indirectly, that is from outside, must be directed towards the wire. In particular, in the case of arcs, the heat generated is used to heat the wire. Direct heating takes place through direct contact between the electrode and the wire.

For safety reasons, the electrical energy source used is preferably a generator for an electrosurgical device, which generator produces a high-frequency alternating current.

For direct heating of a wire, a relatively large current is required. Relatively high means high compared with the maximum current that is usually generated by the generators for commonly used electrosurgical devices. Care should be taken to ensure that, for unipolar application, the current always flows in two, that is both, directions from the contact site of the current conductor into the respective wire.

During unipolar application, as a result of the method used, the current flowing in a stent wire also flows between the stent and the adjacent tissue. If the contact area between the stent and the adjacent tissue is small and/or if the current is strong, the current flowing here can cause thermal damage to the adjacent tissue. This risk increases with the number of stent wires into which current is fed simultaneously.

In order to prevent current being fed into too many stent wires simultaneously, the instrument according to the invention is configured such that current can only be simultaneously fed into a limited number of stent wires and preferably only one wire. This condition must also be observed even if the current available is smaller than the current required to melt several wires simultaneously.

Since the load impedance experienced by the generator and measured between the active electrode and the neutral electrode that is commonly used with the unipolar technique is relatively small on proper use of the instrument according to the invention, the generator of the electrosurgical device must be suitable for operation with low load impedances. Generators of electrosurgical devices have to be short-circuit protected, that is, in the event of a short-circuit between the active electrode and the neutral electrode, they do not suffer damage or fail completely, but this short-circuit protection is implemented in the generators of most electrosurgical devices such that, on occurrence of a short-circuit or if the load impedance falls below a defined minimum, the generator is automatically switched off. Generators for operating instruments according to the invention must be designed and dimensioned such that, even with the smallest of load impedances, such as those which can arise on use of instruments according to the invention, they are not automatically switched off or even destroyed.

For indirect heating of stent wires by electric arcs, the active electrode is equipped with a spacer which is designed such that, when properly used, the active electrode does not directly contact the stent wires, but has a minimum spacing therefrom such that, given a sufficiently high electric voltage between the active electrode and the stent wire, electric arcs are formed which have a temperature sufficiently high that stent wires are thereby heated to their melting point.

The indirect heating of stent wires by electric arcs has the advantage, compared with direct heating, that the electrical energy generated and supplied by the generator is mainly converted to heat in the electric arc whereas, in the case of direct heating, the electrical energy supplied by the generator is mainly converted into heat in the tissue between the stent and the neutral electrode. This results from the distribution of the electrical impedances through which the current must flow between the active electrode and the neutral electrode. During indirect heating, as a rule, it is the electrical impedance of the electric arc and consequently, the heat generation in the arc that predominates. During direct heating, it is the electrical impedance and thus the heat generation between the stent and the neutral electrode—that is, in the tissue—that predominates.

In both direct and indirect heating of stent wires, the circumstance that stent wires which make contact with water-containing tissues generally cannot be heated above the boiling point of water must be taken into account. Instruments according to the invention intended for parting stent wires which make contact with water-containing tissues are therefore equipped with devices for spacing the respective stent wires to be cut from water-containing tissue. This applies both to instruments for direct heating and to instruments for indirect heating of stent wires.

Instruments according to the invention for shortening and/or fragmenting stents consist, in principle, of a rigid or flexible shaft or catheter or comprise a shaft or catheter which can be introduced directly or through instrument channels of rigid or flexible endoscopes into the gastrointestinal tract or the tracheobronchial system or into other hollow organs or corresponding regions such that their distal end reaches to the stent to be shortened and/or fragmented.

At least the electrode device and the protective device comprise, in one embodiment, an effector at the distal end of the instrument. A handle device may be arranged, if needed, at a proximal end of the instrument according to the invention, thereby improving the handling of the respective instrument.

Preferably, the shaft or the catheter is configured as a tube, respectively with a lumen, as a feed apparatus for feeding in a fluid, in particular a gas and/or a liquid, for example, a rinsing liquid, to the electrode of the instrument according to the invention and/or to the hollow organ. In one embodiment, at least the electrode device comprises the feed apparatus, that is, the lumen. With the feeding in of a coolant fluid, it can be prevented, for example, that the active electrode or the whole distal end of the instrument and thus the whole effector becomes overheated, particularly due to electric arcs. At least the distal end can be effectively cooled during operation of the instrument, since the feed apparatus is arranged in a suitable manner relative to the electrode, particularly surrounding it. For this reason, the fastening of the active electrode within the shaft or the catheter is configured such that the coolant is able to flow, in particular, round the active electrode. For example, the electrode has, in part, a helical form so that it can be held in form-fitting manner in the shaft or catheter. As coolant, a gas, for example, air or an inert gas can be used, and said gas can be fed, for example, from the proximal end of the instrument through the shaft or catheter.

When the instruments according to the invention are used close to combustible substances, for example, stents covered with plastics, it may be suitable to introduce an inert gas such as argon via the feed apparatus, particularly into the region of the electric arc. This can be accomplished in the same manner as the introduction of coolant. By this means, undesirable gases situated in hollow organs can also be kept away from the region of action of the arc. In some circumstances, it may therefore be advantageous to generate electric arcs in a protective gas atmosphere (using a protective or inert gas) rather than in air, particularly if combustible material is present in the region of the arc, so that heating of the wire takes place in a protective gas atmosphere.

As explained above, it is advantageous to provide an HF generator to generate the HF current, wherein the current path leads from the HF generator, via a current feed device, to the electrode, and via a neutral electrode and a current return device, back to the HF generator.

The conduction of the electric current between the active electrode and the generator takes place, for example, within the shaft or the catheter, wherein the electric lead between the proximal end of the instrument and the generator is connected either fixed or removably via an electric coupling at the proximal end of the instrument. The current feed device can also be configured such that it is firmly or detachably attached or attachable to a possible handle device.

The generator must be configured such that, on proper use of instruments according to the invention, it supplies the required current or the required voltage therefor. On use of generators which are switched off automatically when the load impedance is too low, an external series impedance of sufficient size or an external matching transformer may be helpful.

In one embodiment, the active electrode comprises a high temperature-resistant material, for example, tungsten, and/or is dimensioned to be, for example, more massive than the wires to be separated, such that it does not melt when properly used.

Provided at the distal end of the instrument or the effector is the protective device which serves to space the stent or a wire of the stent from the tissue of the patient against which it lies or by which it is surrounded. For this purpose, the protective device is advantageously configured electrically insulating and formed from heat-resistant and arc-resistant material. By this means, a selected stent wire can be separated from the tissue in simple manner in order to avoid this stent wire being cooled by the water-containing tissue.

The effector preferably comprises a sleeve or a holder made from electrically non-conductive material, for example ceramic material, for holding the electrode, wherein in one embodiment, the protective device can be firmly connected to the holder, in particular in one piece. As described in greater detail above, the electrode can have a helical region. The electrode is then adapted to the holder by means of the helix in form-fitting manner and thereby substantially securely fixed. The effector is therefore constructed to be extremely stable and easy to use.

Naturally, the instruments according to the invention can also be constructed without any actual lumen, particularly if no fluid has to be or is to be fed to the effector. However, it is advantageous to configure the effector with the holder and thus with a lumen, since in this way, it is ensured that the active electrode is arranged within the holder and cannot make unwanted contact with the tissue.

In one embodiment, the protective device has a device for threading the wire at least into the protective device and/or for separating or distancing the wire from the tissue. This device is preferably configured spatula-shaped, finger-shaped, spoon-shaped or the like such that it can be pushed or pulled between stent wires lying against the tissue and the tissue itself, and far enough until the respective stent wire is accommodated in the protective device and thus lifted off the tissue and positioned for the heating process. Naturally, these spatula-shaped or finger-shaped or similarly formed devices can be adapted in their form and size to the different models of stent existing now and in the future. Devices of this type are manipulated, in particular, in the axial direction of the instrument. Thus the whole instrument can be displaced in the axial direction or the instrument is so configured that only the protective device and/or the device can be manipulated. The effector can also be configured movable per se.

A further embodiment of a device for threading and/or separating and/or distancing stent wires is designed screw-shaped, helical or corkscrew-shaped. By this means, stent wires can be lifted off the tissue in that the device is rotated, that is, screwed, between the stent wire and the tissue.

This device can be optimally adapted in its form, size and manipulation method according to the wire configuration of the respective stent. The most important point is that this device is suitable for distancing the stent wires from water-containing tissue before they are parted, during the direct or indirect heating.

The device is preferably configured such that, using it, a plurality of wires can be threaded and/or separated and/or distanced from the tissue. By this means, relatively large stent fragments can be separated and melted off the stent.

With the instruments according to the invention, the protective device can be configured and arranged relative to the electrode such that the wire can be held at a predetermined distance from the electrode. This enables the formation of an arc in order to cause the wire to melt. The protective device is configured such that the intended distance between the electrode, or a distal end of the electrode, and the threaded wire is assured.

In one embodiment, the protective device or the effector has at least one guide which is configured such that when the instrument is pressed against the tissue and/or the stent and/or when the device and/or the instrument is pushed or rotated, the wire slips into the guide and can be fixed therein. By this means, the wire can be easily and reliably positioned relative to the active electrode. If the guide is configured as at least one notch, the wire can be easily accommodated in this notch. The guide, in particular the notch, advantageously has one region in which the wire can be positioned in an end position for safe processing by means of the active electrode.

According to the invention, the guide can be oriented and/or dimensioned relative to a distal end of the electrode such that the distal end touches the wire and can thereby conduct a current directly into it. Preferably, the electrode and its holder formed by the helix are elastically constructed such that a reliable contact is produced without the operator having to operate the instrument too precisely.

It is also possible to configure the arrangement and/or dimensioning of the guide relative to the end of the electrode such that the defined distance between the wire, particularly in its end position in the guide, and the distal end of the electrode is maintained. In order to ensure the distance required for generating electric arcs between the stent wires to be parted and the active electrode, the effector is accordingly equipped with a spacer. This spacer is also made from electrically non-conductive, heat-resistant and arc-resistant material. In principle, therefore, the guide is configured such that the wire which has been taken up can be held at the predetermined distance from the electrode. The protective device also ensures, with the guide, that at the site where it is to be parted, the wire positioned in the guide does not touch any water-containing tissue.

Preferably, the instrument according to the invention is configured with at least one movement device such that at least one partial region of the effector is movable in controlled manner for its positioning. Reference is made in this regard to WO 97/11647 wherein a distal end of a tube is also tiltable, i.e. bendable, out of an endoscope relative to an exit direction or an axial direction of the instrument. If, in the instrument according to the invention, the effector is additionally movable, for example, relative to the rest of the shaft or catheter, this simplifies the positioning of the active electrode, and the take-up of the stent wire or a plurality of wires can be more easily carried out. In order to ensure the mobility of the effector, that is, of at least partial regions of the distal end of the instrument, the movement device has an elastically deformable device. This may, for example, be provided as a flexible bellows (expansion bellows) and is arranged such that, for example, the protective device is movable for easier threading of the relevant wire.

A user can bring about the movement of at least partial regions of the effector, preferably by means of a manipulator which, in an advantageous embodiment is configured, for example, as a cable element or as a rod element. The manipulator is thus connected to the effector such that the effector is movable on actuation of the manipulator. If the instrument is guided via an instrument channel of an endoscope, the manipulator can be guided via another instrument channel. The user actuates the manipulator and thereby achieves orientation, that is, bending or tilting, of the relevant effector region relative to the axial direction of the instrument and possibly also a return to the straight orientation.

The protective device of instruments according to the invention can preferably comprise a holding device for firmly holding the wire, a stent fragment or the stent. This means that a device is provided which, for example, prevents slipping of a wire, once it has been taken up or threaded in, out of the protective device or the device for threading and/or separating and/or distancing the wire from the tissue. For this purpose, the protective device can comprise at least one barb as the holding device, which ensures a secure hold of the wire in the protective device. Thus with the barb, wires can be “caught” and pulled away from the tissue.

The holding device preferably has a plurality of barbs which are arranged spaced essentially evenly from one another on the protective device for reliable take-up of the wire, the stent fragment or the stent (even on imprecise manipulation of the instrument or device). If the effector has, for example, a circular cross-section, the barbs are preferably arranged radially symmetrically.

According to the invention, the holding device can be arranged on the device for threading and/or separating and/or distancing the wire from the tissue. The holding device supports the protective device or the device.

It may be advantageous to configure the holding device for moving the wire, the stent fragment or the stent to be movable itself. The barb would then, for example, be movable relative to the protective device and could be brought in the direction towards the guide. This would also simplify the positioning of the wire, the stent fragment or even the stent.

If the wire, the stent fragment or the stent can be firmly held by means of the holding device, it can be removed in controlled manner out of the operation region, that is, withdrawn from the hollow organ.

The movement device and the holding device are arranged on the instrument such that these also preferably comprise the effector.

Preferably, the device for threading and/or separating and/or distancing is configured such that it is movable relative to the shaft or catheter, preferably in a guide device arranged on the instrument. In this event, the device would not be connected in one piece with the holder or the sleeve, but movable relative thereto, for example, laterally thereon. The guide device could then be arranged, for example, on the holder and accommodate the device. The device for threading and/or separating or distancing could then be moved, for example, by means of a manipulator as described above and the wire or a plurality of wires that have been taken up could be brought to the active electrode. If the separately guided device is configured, for example, as a hook element, it is preferably movable back and forth in the axial direction of the instrument and can thus take up at least one stent wire.

Using the instruments according to the invention, stents can be fragmented, that is trimmed, in hollow organs and the fragments can be removed from the hollow organ. Stents can also be removed in toto from the hollow organ, particularly if the instruments are configured with the holding device described above.

From the standpoint of the method, the object of the invention is thereby achieved that, in a method for endoscopically controlled shortening and/or fragmentation of stents made from electrically conductive material and situated in the gastrointestinal tract, the tracheobronchial system or in other hollow organs, with an instrument comprising an electrode device with an electrode and a protective device which is mechanically connected to the electrode device, the following steps are provided:
a) introducing the instrument into the hollow organ as far as the stent;
b) separating and/or distancing at least one wire from the tissue of the gastrointestinal tract, tracheobronchial system or other hollow organ by pushing in or screwing in the protective device between the wire and the tissue and positioning the at least one wire at least close to the electrode by means of the protective device such that an HF current can be conducted via the electrode into the wire and/or electric arcs can be formed between the electrode and the at least one wire;
c) introducing the HF current into the at least one wire by means of the electrode and/or forming electric arcs between the electrode and the at least one wire and parting the wire by heating and melting the wire;
d) repeating steps b) and c) to shorten and/or fragment the stent.

By means of this method, using the instruments according to the invention, at least one stent wire can be melted and thereby detached from the stent. In order to melt a plurality of wires of the stent positioned in the hollow organ off the stent and thereby to shorten, trim or fragment the stent, or even to remove the whole stent, steps b) and c) need to be repeated accordingly often.

In another embodiment it is also provided that a stent fragment or the stent is removed from the gastrointestinal tract, tracheobronchial system or other hollow organ. This addresses the problem of complete removal from the human body. If the stent fragment removed from the stent with the instruments according to the invention is simultaneously removed from the region of deployment, a fragment, once separated from the stent, does not have to remain in the hollow organ until its removal from the region by another instrument, for example, forceps. The instrument is configured such that complete removal of the fragment can be carried out therewith.

As described in detail above, the HF current can be introduced into the wire being processed by direct contact between the electrode and the wire or via an electric arc. The formation of an arc is usually preferable since the respective wire is indirectly heated by the heat of the arc and is eventually melted. A relatively small current is needed to form an electric arc. Furthermore, the conversion of electrical energy into heat takes place in focussed manner, that is, the heat of the arc can be essentially used where it is needed.

Since instruments according to the invention are preferably configured as a rigid or flexible shaft or catheter or comprise a shaft or a catheter, in one embodiment, the instrument is guided to the stent directly or through an instrument channel of a rigid or flexible endoscope, so that a distal end of the instrument reaches to the stent to be shortened and/or fragmented.

Preferably, by means of the arrangement of at least the electrode device and the protective device, an effector is formed, in principle, at the distal end of the instrument or as the distal end. By means of the effector, the desired effect is brought about at the wire being processed.

In one embodiment, an instrument is used which comprises, at a proximal end, a handle device for handling the instrument. This simplifies the operation of the instrument.

In a further embodiment, feeding in of a fluid, in particular a gas and/or a liquid, for example a rinsing liquid, to the electrode and/or the hollow organ via a lumen of the shaft or the catheter configured as a feed apparatus is provided. The feeding in of fluids can serve many different purposes. For example, the active electrode or the whole effector can be cooled by a coolant fluid, particularly if these parts become too hot due to the formation of arcs. Furthermore, the heating of the wire can take place with the introduction of a protective gas, such as argon, under a protective gas atmosphere, so that combustible gases situated in the hollow organs are kept away from the region of action of the arcs. In this way, an instrument is used in which the electrode device itself comprises the feed apparatus.

In another embodiment, an HF generator is used to generate the HF current, wherein the current path preferably leads from the HF generator via a current feed device to the electrode and, via a neutral electrode and a current return device, back to the HF generator. The use of high frequency current offers a high level of safety for the patient.

In another embodiment, an instrument is used on which the current feed device is or can be firmly or detachably connected to the shaft or the catheter and/or the handle device. This facilitates operation of the instrument.

As the electrode material, preferably a high temperature-resistant material is used, for example tungsten, so that melting of the electrode is avoided when properly used. In addition, it may be useful to use a suitably dimensioned electrode in order to prevent its destruction.

One embodiment provides that an instrument is used wherein the protective device is suitably configured at the distal end of the instrument or the effector. A protective device is herein used which is made from electrically insulating and/or heat-resistant and arc-resistant material. This facilitates use of the instrument and enables effective operation. The protective device is therefore protected against abrasion and heat is not conducted into surrounding tissues.

Preferably, an instrument is used wherein the effector comprises a sleeve or a holder to hold the electrode. In one embodiment, the holder is made of electrically non-conducting material, for example, ceramic material. Thus the effector is configured abrasion-resistant and prevents heat conduction into surrounding tissues. On use of a protective device connected, particularly in one piece, to the holder, the protective device is easily operated via the movement of the instrument. The effector is configured to be extremely robust and easy to operate and the method steps can be carried out very easily.

If the protective device has a device for threading the wire into the protective device and/or for separating or distancing the respective wire from the tissue of the hollow organ, then the wire is preferably taken up, that is threaded, in simple manner with this device into the protective device and thereby lifted off, that is separated or distanced from, the tissue. By this means, the wire can be safely and easily positioned in the correct position relative to the active electrode and reliably heated and parted. The device is therein pushed or pulled in a substantially straight-line movement in the axial direction of the instrument under the wire, that is, between the wire and the tissue, so that the wire is appropriately positioned.

If the device of the protective device is configured screw-shaped or corkscrew-shaped, it is screwed in an essentially twisting or rotary movement under the wire or under a stent fragment. What is important herein is that this device is suitable for distancing from the tissue the stent wires to be parted during the direct or indirect heating.

Preferably, by means of this device, a plurality of wires is threaded and/or separated and/or distanced from the tissue simultaneously, so that relatively large stent fragments can also be parted from the stent.

It is necessary, for the formation of arcs, that a particular distance is provided between the active electrode, in particular a distal end of the active electrode, and the wire to be heated. For that purpose, an instrument is preferably used wherein the protective device is configured such that the wire or wires taken up can be held by it at the desired distance. In one embodiment of the method, it is provided that the instrument is pressed (for example, against the stent and/or the tissue) and/or the instrument and/or the device for threading and/or for separating and/or distancing is pushed or pulled and/or rotated such that the wire slips into at least one guide, in particular at least one notch and is fixed therein. As described above in greater detail, for actuation of the device, the whole instrument can, in principle, be moved in corresponding manner or the device and/or the protective device can be manipulated accordingly.

In another embodiment, an instrument is used wherein the wire that has been threaded and/or separated and/or distanced from the tissue is held by means of the guide at the predetermined distance from the electrode. It is also possible, however, to use an instrument with a guide which is configured such that touching of the electrode by the wire introduced into the guide is enabled.

In a further embodiment, it is provided that at least a partial region of the effector is moved in controlled manner by means of a movement device configured on the instrument. Movement of the effector is understood herein to mean orienting, that is bending or tilting of the relevant effector region in relation to the axial direction of the instrument and possibly also return to the straight orientation. For this purpose, the movement device has an elastically deformable device, preferably a bellows, wherein the effector (or at least the partial region) is moved via the elastically deformable device. This means that the movement can only be performed by means of the elastically deformable element. In order for a user to move the effector or at least partial regions thereof, a manipulator which comprises the movement device is preferably actuated. The manipulator is preferably configured as a cable element or a rod element, so that, in one embodiment, by means of its actuation, that is the actuation of the manipulator, the effector is moved by means of the elastically deformable device. The mobility of the effector facilitates the carrying out of the method, since the protective device and thus the cable can be easily and reliably positioned in relation to the active electrode. In addition, it is made possible with a manipulable effector that the whole endoscope or instrument does not have to be moved and it may suffice to move just the effector in the hollow organ or at the operating site and to orient it accordingly.

In another embodiment, the wire, the stent fragment or the stent is firmly held by a holding device configured on the protective device. Through the possibility of firmly holding the wire, the stent fragment or the stent, the shortening and/or fragmentation of stents can be carried out reliably and easily, since the slipping out of a stent component part, once taken up, is prevented. Preferably, the wire, the stent fragment or the stent is firmly held by at least one barb as the holding device or by a plurality of barbs, which, for secure take-up of the wire, the stent fragment or the stent, in one embodiment, are essentially arranged evenly spaced from one another on the protective device. If a plurality of barbs is used, a wire can be more easily “caught”, even if the instrument and/or the protective device is not moved in targeted manner. In another embodiment, an instrument is used wherein the holding device is configured on the device for threading and/or separating and/or distancing the wire. Once a wire or a fragment has been taken up, it can be removed from the hollow organ with the holding device, since said holding device firmly holds the wire (the same applies for the stent in toto). The holding device serves therefore as a salvaging device for the melted off wire or even the stent. It is thus possible to dispense with an additional instrument for removing a fragment from the hollow organ.

In a further embodiment, the wire, the stent fragment or the stent is firmly held by means of at least one movable holding device to move the wire, the stent fragment or the stent. The barb would then be moved, for example, relative to the protective device and could be brought in with the grasped wire component in the direction of the guide.

In another embodiment, it is provided that the device for threading and/or separating and/or distancing is moved in a guide device arranged on the instrument, for example, in the axial direction of the instrument, relative to the shaft or catheter. Since the device is not connected in one piece to the holder in this case, it can be moved laterally on and relative to it. In another embodiment, the device for threading and/or separating and/or distancing can be moved in and relative to said holder. In one embodiment, the device for threading and/or separating and/or distancing can be moved by means of a manipulator.

The invention will now be described in greater detail based on exemplary embodiments and making reference to the drawings, in which:

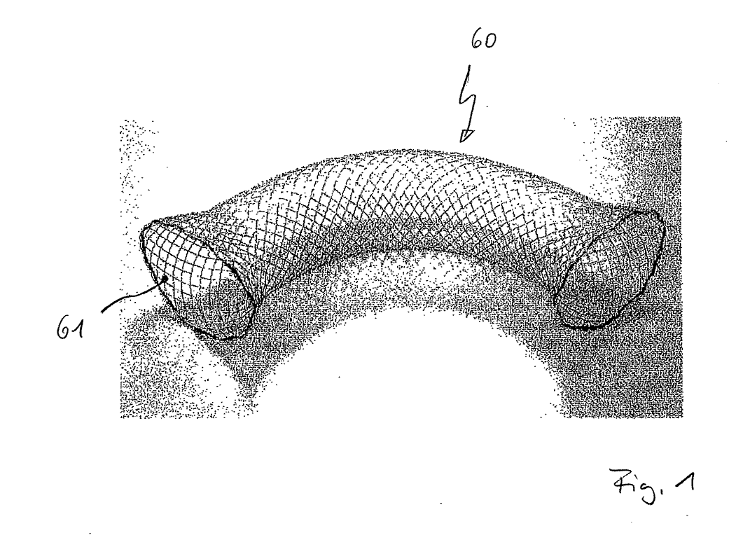

FIG. 1 shows an example of a stent;

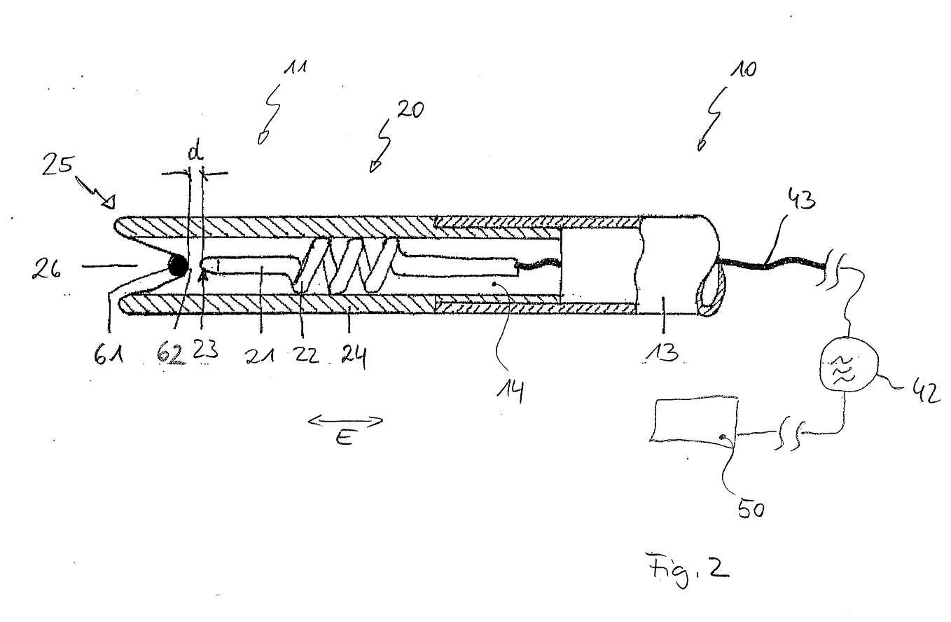

FIG. 2 shows a cross-section through an effector at the distal end of an instrument according to the invention;

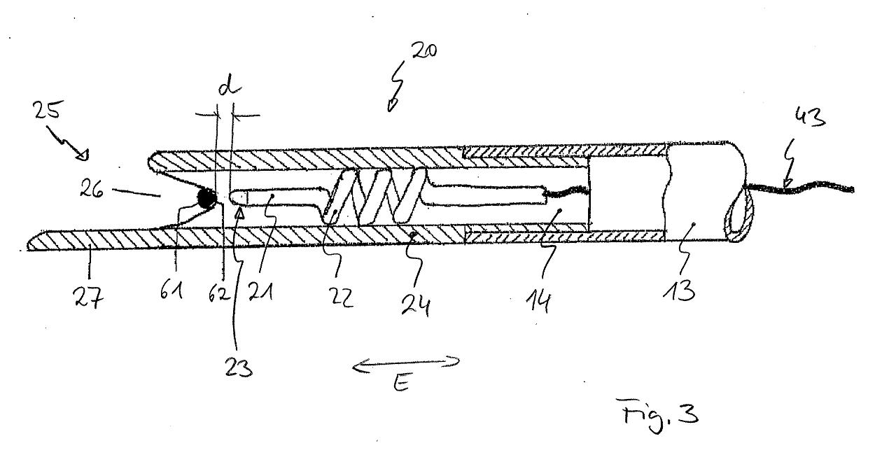

FIG. 3 shows a cross-section through an effector at the distal end of an instrument according to the invention comprising a spatula-shaped device for distancing stent wires from water-containing tissue;

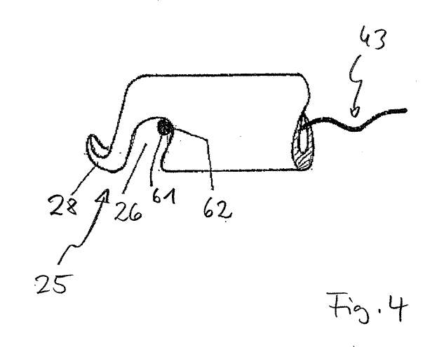

FIG. 4 shows an effector at the distal end of an instrument according to the invention comprising a corkscrew-shaped device for distancing stent wires from water-containing tissue;

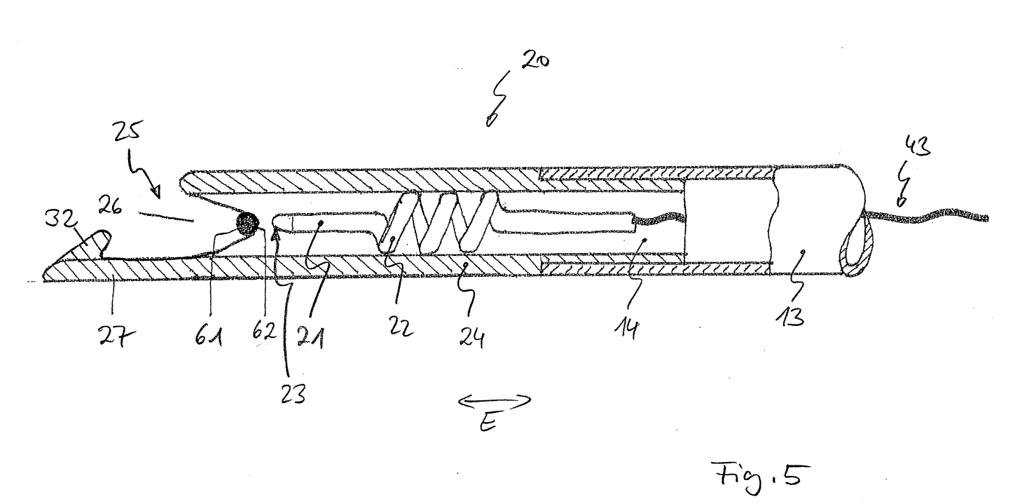

FIG. 5 shows a cross-section through an effector at the distal end of an instrument according to the invention comprising a spatula-shaped device for distancing stent wires from water-containing tissue and a holding device for holding the stent wires;

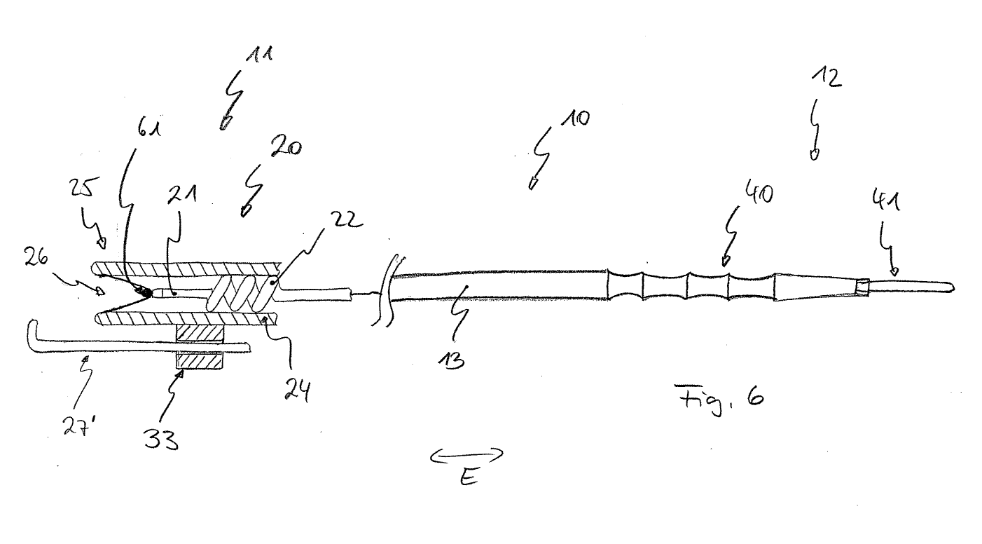

FIG. 6 shows a portion of an instrument according to the invention comprising a specially configured device for distancing stent wires from tissue at the distal end of the instrument and a handle device at the proximal end; and

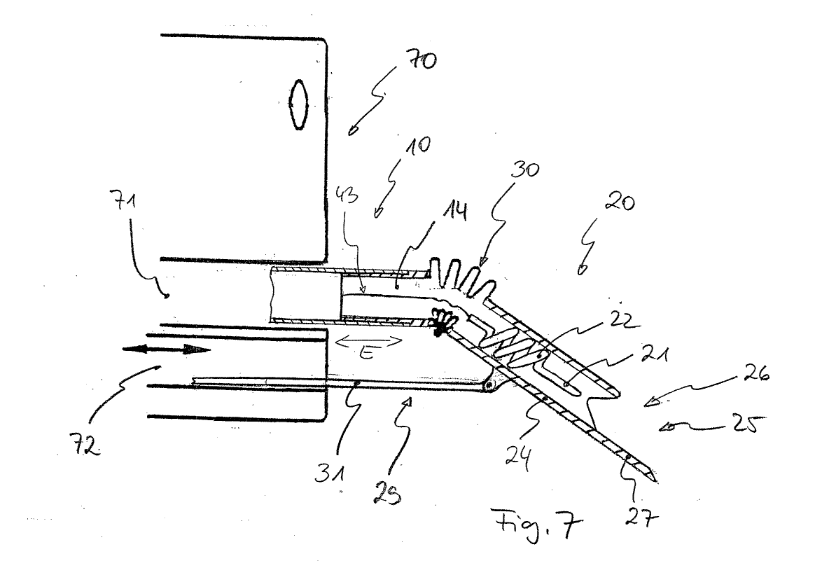

FIG. 7 shows a cross-section through an effector at the distal end of an instrument according to the invention guided in an endoscope, comprising a movement device for moving the effector.

FIG. 1 shows an example of a stent 60. Metal stents are elastic tubes braided, knitted or produced by other means from special metal wires, and comprising meshes of differing sizes. The purpose of stents of this type is to expand the lumen of hollow organs, for example, the esophagus, which are pathologically constricted as a result of a stenosing tumour growth, by means of their radially acting elastic force. Stents, and in this case, particularly metal stents fulfil their function only and for as long as they keep the lumen that is required for the functioning of the respective organ free. If a stent does not fulfil its purpose, it may be necessary to remove it from the hollow organ concerned. However, this can be very difficult. If the stent lies too firmly against the organ wall or if tissue has grown into its mesh and/or if the stent is deformed so that it cannot be removed in one piece, it must be divided into sufficiently small removable fragments, for which purpose the instruments according to the invention can be used. With these instruments, the stent wires are heated along the respective planned parting lines such that they melt.

FIG. 2 shows an embodiment of the instrument 10 according to the invention in longitudinal section, wherein the part which is of importance here is designated the effector 20 and is arranged at the distal end 11 of a rigid or flexible shaft or catheter 13. The effector 20 comprises an electrode 21 with a distal end 23, which is fixed by a helix 22 within a lumen 14 of the effector 20. The electrode 21, which is connected via a supply lead 43 to an HF generator 42 is hereinafter designated the active electrode 21 in order to make clear the difference from a neutral electrode 50, via which the generator 42 is connected in electrically conductive manner to the tissue of the patient.

The effector 20 comprises a sleeve 24 made from electrically non-conductive material, for example, ceramic material.

Provided at the distal end of the sleeve 24 or of the effector 20 is a protective device 25 which serves to separate the stent 60 or a wire 61 of the stent 60 from the tissue of the patient on which it lies or by which it is surrounded. This protective device 25 also has a guide 26 which, in the exemplary embodiment shown in FIG. 2, is configured notch-like such that a stent wire 61 slips into the notch or the guide 26 and is fixed at its base when the instrument 10 is pressed forward.

In an embodiment of the invention shown in FIG. 6, the guide 26 is oriented and dimensioned relative to the distal end 23 of the electrode 21 such that the distal end 23 touches the wire 61 and therefore a current can be conducted into said wire. Preferably, the electrode 21, together with its holder comprising the helix 22 is herein elastically configured such that a reliable contact is formed without the operator having to handle the instrument too precisely.

In the embodiments shown in FIGS. 2 and 3, the arrangement and dimensioning of the guide 26 relative to the end 23 of the electrode 21 is carried out such that a defined distance d remains between the wire 61 in its end position 62 in the guide 26 and the distal end 23 of the electrode 21. In other words, for direct heating of stent wires as per the general description of the invention above, the distance d between the end position 62 of the guide 26 and the distal end 23 of the active electrode 21 is zero or even negative, that is, such that a stent wire 61 situated in the end position contacts the active electrode 21, or is pressed against the active electrode, in electrically conductive manner.

For indirect heating of stent wires according to the above general description of the invention, the distance d between the end position 62 of the guide 26 and the distal end 23 of the active electrode 21 is greater than zero, and such that electric arcs can form between a stent wire 61 situated in the end position 62 and the active electrode 21 when a sufficiently large electric voltage is applied between the stent wire and the active electrode.

The sleeve 24 provides, on the one hand, the protective device 25 which ensures that, at the site to be parted, the wire 61 positioned in the end position 62 of the guide 26 does not touch any water-containing tissue. On the other hand, this sleeve 24 provides a sleeve-shaped holder for the electrode 21.

Since operators generally see the effector 20 from the proximal direction and consequently have no direct view of the distal end of the effectors 20 and since it can also be difficult to take up into the guide 26 stent wires lying close to tissue, it is suitable additionally to have a device 27, 28 for threading these stent wires into the guide 26 or more generally into the protective device 25 at the distal end of the sleeve 24, for example according to FIG. 3 or FIG. 4.

An exemplary embodiment of a device for threading stent wires into the guide 26 is shown in section in FIG. 3. This device 27 is spatula-shaped, finger-shaped or similarly configured such that this device can be pushed between stent wires lying against the tissue and the tissue itself, and far enough until the respective stent wire 61 has reached the end position 62 in the guide 26. Naturally, these spatula-shaped or finger-shaped or similarly configured devices can be adapted in their form and size to the various existing and future models of stent. Devices according to FIG. 3 are manipulated, in particular, in the axial direction of the instrument.

Another exemplary embodiment of a device for threading stent wires into the guide 26 is shown in FIG. 4. This device 28 is configured helical or corkscrew-shaped. In this way, stent wires 61 can be taken up into the guide 26 by rotation of the instrument (possibly also only the device) and brought into the end position 62.

In order to prevent the active electrode 21 and the whole effector 20 becoming overheated, in particular, by electric arcs, the shaft or catheter 13 may be a tube or pipe through which a suitable gaseous or liquid coolant can be introduced from the proximal to the distal end. For this reason, the fastening of the active electrode 21 within the sleeve 24 of the effector 20 is designed such that a coolant can effectively cool, in particular, the active electrode 21. For example, the electrode 21 is fastened in the holder 24 by means of a form-fitting helix 22.

If these instruments are used in the vicinity of combustible substances, for example, stents coated with plastics, it may be suitable to introduce an inert gas, for example argon, particularly in the region of the electric arc. This can be carried out in a similar manner to the introduction of coolants.

FIG. 5 shows a cross-section through an effector 20 at the distal end of an instrument according to the invention in a further embodiment. This embodiment corresponds essentially to that shown in FIG. 3. The spatula-shaped device 27, additionally has a holding device 32 for holding one or more stent wires 61, and possibly even to hold the whole stent 60. By this means, on the one hand, slipping out of a wire once it has been taken up is prevented and, on the other hand, the holding device can be configured such that the wire 61, when melted, can be removed with the instrument 10 from the hollow organ. FIG. 5 shows that the holding device 32 is configured as a barb. Said barb is arranged on the spatula-shaped device 27 and thus facilitates the positioning of the wire 61.

In an embodiment not shown here, the protective device 25 can have a plurality of barbs 32 in order to ensure secure holding and possibly removal of the wire 61, the stent fragment or the stent. A radially symmetrical arrangement enables a wire 61 to be “caught”, independently of the manipulation of the instrument and to be taken up into the protective device 25, in particular, into the guide 26.

It may possibly be advantageous to configure the holding device 32 movable itself (not shown) for moving the wire 61, the stent fragment or the stent. Thus the wire, once taken up, can be moved in the direction towards the electrode 21 and could thus be taken up by simple means, for example, into the guide 26.

With the holding device, it is essentially possible to remove the stent fragment or the stent completely out of the hollow organ and thus out of the patient. If the wire is salvaged by the holding device, no further instruments are needed to remove the wire from the hollow organ.

FIG. 6 shows a portion of an instrument 10 according to the invention with a separately guided device 27′ for distancing stent wires from tissue at the distal end 11 of the instrument 10 and a handle device 40 at the proximal end 12. The effector 20 is configured, in principle, as shown in FIG. 2. However, the guide 26 is provided such that the wire 61 positioned therein comes into direct contact with the electrode 21 or the distal end 23 of the electrode 21, so that direct heating of the wire is enabled. The embodiment shown here also differs in having a separately guided device 27′ for threading and/or separating and/or distancing, wherein said device 27′ is provided in a guide device 33 arranged on the holder 24 or the sleeve and can therefore be moved in an axial direction E of the instrument 10 relative thereto. The device 27′ for threading and/or separating and/or distancing is configured here as a hook element and can be moved, for example, via a manipulator (in principle an extension of the hook element). This means that the device 27′ is independently movable so that a wire 61 can be taken up into the protective device 25 without explicit movement of the instrument 10. The guide device 33 can be provided in one piece with the holder 24 or as a discrete component thereon.

Provided at the proximal end 12 of the instrument 10 is the handle device 40, which facilitates the handling of the instrument 10. Also configured on the handle device 40 is a current connection element 41, so that the supply lead 43, that is, the current feed device can be connected to the handle device 40 and thereby to the shaft or the catheter 13.

FIG. 7 shows a cross-section through an effector 20 at the distal end 11 of an instrument 10 according to the invention guided in an endoscope 70 and comprising a movement device 29 for moving the effector 20. The instrument 10 is thus guided to the relevant hollow organ via the endoscope 70 which has a plurality of channels 71, 72. By means of the movement device 29, at least one partial region of the effector 20 can be moved in controlled manner without the endoscope 70 or the entire instrument 10 having to be moved. Movement of the effector means, in this case, orientation, that is bending or tilting of the relevant effector region relative to the axial direction E of the instrument and possibly also its return into the straight orientation. For this purpose, the effector 20 is configured with an elastically deformable device 30, for example, a bellows which ensures, on the one hand, sufficient stiffness and, on the other hand permits bending of the effector 20 away from the straight orientation at a site provided therefor. In order that a user can bring about the desired orientation of at least one distal end of the effector 20, the effector 20 is connected to a manipulator 31, in this case a rod element, wherein the rod element articulates on the effector 20 and can be actuated via a further channel 72 of the endoscope 70. The user actuates the manipulator 31 in a direction shown by the arrow and thereby achieves the desired orientation of the effector 20 or at least partial regions of the effector, for example, the device 27′, 28, relative to the axial direction E of the instrument. The instrument can naturally also be configured such that it can be used without the aid of an endoscope.

Alternatively, it is possible, in place of a rod element, to use a cable element, although the effector 20 can only be tilted in one direction, whereas return into the starting orientation, for example to the straight orientation, is made more difficult. The cable element can also be guided through another channel of the endoscope or placed within the channel in which the instrument itself is arranged.

The instruments according to the invention described here are fed into the hollow organ as far as the stent, and then at least one wire or stent fragment is separated and/or distanced by the protective device from the tissue of the gastrointestinal tract, tracheobronchial system or other hollow organ by pushing or screwing in the protective device between the wire and the tissue. If the protective device has a device for threading and/or separating and/or distancing, the wire can be reliably and efficiently hooked in and threaded into the protective device, in particular into the guide in that the instrument or even only the effector is manipulated in appropriate manner. The wire can thus be positioned in the protective device and in the effector such that the wire can be melted with the aid of the electrode and parted from the stent. By means of a holding and movement device, the positioning of the wire can be facilitated in that, on the one hand, the wire taken up is securely fixed by the holding device and possibly even completely removed from the hollow organ and the human body. On the other hand, by means of the movement device, manipulation of the effector is carried out so that, without explicit movement of an endoscope that may be used, or of the whole instrument, targeted take-up of a wire can take place.

It should be noted in conclusion that this instrument is a unipolar instrument for whose use, naturally, a neutral electrode has to be placed on the patient and, furthermore, a generator is required, in particular a generator of an electrosurgical device and the obviously required cables and plug connections for connecting the active electrodes and the neutral electrodes to the generator and switches for activating the generator. A description of these elements, which are generally known to persons skilled in the art in this field, is not given here. Nevertheless, the generator to be used for operating the instruments according to the invention must make available the current necessary for direct heating of stent wires and the voltage necessary for indirect heating of stent wires.

Reference numbers

| | |
|---|---|
| 10 | Instrument |
| 11 | Distal end of the instrument |
| 12 | Proximal end of the instrument |
| 13 | Catheter, shaft |
| 14 | Lumen |
| 20 | Effector |
| 21 | Active electrode |
| 22 | Helix |
| 23 | Distal end of the electrode |
| 24 | Sleeve-shaped holder |
| 25 | Protective device |
| 26 | Guide |
| 27 | Device |
| 27′ | Device |
| 28 | Device |
| 29 | Movement device |
| 30 | Elastically deformable device |
| 31 | Manipulator |
| 32 | Holding device |
| 33 | Guide device |
| 40 | Handle device |
| 41 | Current connection element |
| 42 | HF generator |
| 43 | Supply lead, current feed device |
| 50 | Neutral electrode |
| 60 | Stent |
| 61 | Stent wire |
| 62 | End position |
| 70 | Endoscope |
| 71 | First instrument channel |
| 72 | Second instrument channel |
| d | Distance |
| E | Axial direction of the instrument |

1. An instrument for endoscopically controlled shortening or fragmentation of stents made from electrically conductive material situated in hollow organs, comprising
an electrode device with an electrode for introducing a high-frequency current into at least one wire of the stent and/or for forming electric arcs between the electrode and the at least one wired; and
a protective device which is configured and mechanically connected to the electrode device such that the at least one wire can be thereby separated or distanced from the tissue of the hollow organ during introduction of the high-frequency current or formation of electric arcs.

2. The instrument according to claim 1,
wherein the electrode device is configured such that the at least one wire can be heated directly by means of the high-frequency current by contact between the electrode and the at least one wired.

3. The instrument according to claim 1,
wherein the electrode device is configured such that the at least one wire can be heated indirectly by formation of electric arcs between the electrode and the at least one wire.

4. The instrument according to claim 1,
wherein the instrument comprises a rigid or flexible shaft or catheter, wherein the shaft or catheter can be guided directly or through an instrument channel of a rigid or flexible endoscope to the stent.

5. The instrument according to claim 1,
wherein at least the electrode device and the protective device are arranged as an effector at a distal end of the instrument.

6. The instrument according to claim 1,
wherein configured at a proximal end of the instrument is a handle device for handling the instrument.

7. The instrument according to claim 4,
wherein the shaft or the catheter is configured as a tube or a hose, respectively, with a lumen as a feed apparatus for feeding in a fluid to the electrode or to the hollow organ.

8. The instrument according to claim 7,
wherein the feed apparatus is arranged to surround the electrode such that the electrode or the electrode device or the whole effector can be cooled by the fluid fed in.

9. The instrument according to claim 7,
wherein the fluid is a protective gas or an inert gas which can be fed in via the feed apparatuses so that heating of the at least one wire takes place in a protective atmosphere.

10. The instrument according to claim 1,
wherein a high-frequency generator is provided for generating the high-frequency current, wherein the current path leads from the high-frequency generator via a current feed device to the electrode and via a neutral electrode and a current return device back to the high-frequency generator.

11. The instrument according to claim 10,
wherein the current feed device is configured to be firmly or detachably connected or connectable to the shaft or the catheter or the handle device.

12. The instrument according to claim 1,
wherein the electrode is made from a high temperature-resistant material.

13. The instrument according to claim 1,
wherein the protective device is configured at the distal end of the instrument.

14. The instrument according to claim 1,
wherein the protective device is electrically insulating.

15. The instrument according to claim 1,
wherein the protective device is made from heat-resistant and electric arc-resistant material.

16. The instrument according to claim 5,
wherein the effector comprises a sleeve or a holder for holding the electrode.

17. The instrument according to claim 16,
wherein the protective device and the holder for holding the electrode are one piece with it.

18. The instrument according to claim 1,
wherein the protective device comprises a device for threading the at least one wire into the protective device and for separating or distancing the at least one wire from the tissue.

19. The instrument according to claim 18,
wherein the device is configured such that therewith a plurality of wires can be simultaneously threaded in and separated or distanced from the tissue.

20. The instrument according to claim 18,
wherein the device is configured as one of a spoon-shaped, a finger-shaped or a spatula-shaped device such that the device can be pushed or pulled in a substantially straight-line movement in an axial direction of the instrument under the at least one wire.

21. The instrument according to claim 18,
wherein the device is configured screw-shaped or corkscrew-shaped such that the device can be screwed and/or pushed under the at least one wire with an essentially twisting or rotating movement.

22. The instrument according to claim 1,
wherein the protective device is configured and arranged relative to the electrode such that the at least one wire can be held at a predetermined distance from the electrode.

23. The instrument according to claim 22,
wherein the protective device is configured such that the distance for formation of electric arcs is provided between the electrode and the threaded wire.

24. The instrument according to claim 1,
wherein the protective device comprises at least one guide which is configured such that when the instrument is pressed forward or when the device or the instrument is pushed or rotated, the at least one wire slips into the guide and can be fixed therein.

25. The instrument according to claim 24,
wherein the guide is configured as at least one notch, so that the at least one wire can be accommodated in the notch.

26. The instrument according to claim 5,
wherein the guide is configured such that the wire taken up can be held at a predetermined distance from the electrode.

27. The instrument according to claim 5,
wherein the instrument is configured with at least one movement device such that at least one partial region of the effector can be moved in a controlled manner.

28. The instrument according to claim 27,
wherein the movement device Comprises an elastically deformable device for moving the at least one partial region of the effector.

29. The instrument according to claim 28,
wherein the elastically deformable device is configured as a bellows.

30. The instrument according to claim 29,
wherein the movement device comprises a manipulator which is connected to the effector such that at least one partial region of the effector is movable, via the elastically deformable devices, on actuation of the manipulators.

31. The instrument according to claim 30, wherein the manipulator is configured as a cable element.

32. The instrument according to claim 1,
wherein the protective device comprises a holding device for firmly holding the at least one wire, a stent fragment or the stent.

33. The instrument according to claim 32,
wherein the holding device is configured as at least one barb.

34. The instrument according to claim 32,
wherein the holding device comprises a plurality of barbs, which are arranged essentially evenly spaced from one another on the protective device (for reliable take-up of the at least one wire, the stent fragment or the stent.

35. The instrument according to claim 32,
wherein the holding device is configured on a device for threading the at least one wire into the protective device.

36. The instrument according to claim 32,
wherein the holding device is configured movable for moving the at least one wire, the stent fragment or the stent.

37. The instrument according to claim 32,
wherein the holding device (32H is configured such that the stent fragment or the stent can be removed from the hollow organ by means of the holding device.

38. The instrument according to claim 18,
wherein the device for threading and separating or distancing is configured such that the device is movable relative to a shaft or catheter of the instrument.

39. The instrument according to claim 38,
wherein the device is configured such that it is movable in a guide device arranged on the instrument.

40. A method for endoscopically controlled shortening or fragmentation of stents made from electrically conductive material and situated in a hollow organ with an instrument which includes
an electrode device with an electrode and
a protective device which is mechanically connected to the electrode device,
wherein the method comprises:
introducing the instrument into the hollow organ as far as the stent;
separating or distancing at least one wire from the tissue of the hollow organ by pushing in or screwing in the protective device between the at least one wire and the tissue and positioning the at least one wire at least close to the electrode by means of the protective device such that a high-frequency current can be conducted via the electrode into the at least one wire or electric arcs can be formed between the electrode and the at least one wire;
introducing the high-frequency current into the at least one wire by means of the electrode or forming electric arcs between the electrode and the at least one wire and parting the at least one wire by heating and melting the wire; and
repeating the separating and introducing of high-frequency current steps to shorten or fragment the stent.

41. The method according to claim 40,
further comprising removing a stent fragment or a stent by means of the instrument from the hollow organ.

42. The method according to claim 40
wherein the high-frequency current is introduced into the at least one wire by contact between the electrode and the at least one wire for direct heating of the at least one wire.

43. The method according to claim 40,
wherein electric arcs are formed between the electrode and the at least one wire for indirect heating of the at least one wire.

44. The method according to claim 40, further comprising guiding a rigid or flexible shaft or catheter of the instrument to the stent directly or through an instrument channel of a rigid or flexible endoscope.

45. The method according to claim 40, wherein, by means of the arrangement of at least the electrode device and the protective device, an effector is formed.

46. The method according to claim 40, wherein an instrument is used which comprises, at a proximal end, a handle device for operating the instrument.

47. The method according to claim 44, further comprising feeding in a fluid to the electrode or the hollow organ via a lumen of the shaft or the catheter configured as a feed apparatus.

48. The method according to claim 47,
wherein the fluid is fed in via the feed apparatus so that the electrode or the electrode device is cooled by the fluid fed in.

49. The method according to claim 47,
wherein the fluid fed in is a protective gas or an inert gas, so that heating of the at least one wire takes place in a protective gas atmosphere.

50. The method according to claim 40, wherein a high-frequency generator his used to generate the high-frequency current, and
wherein the current path leads from the high-frequency generator via a current feed device to the electrode and, via a neutral electrode and a current return device, back to the high-frequency generator.

51. The method according to claim 50,
wherein an instrument is used on which the current feed device is or can be firmly or detachably connected to a shaft or catheter or handle device of the instrument.

52. The method according to claim 40, wherein an electrode is used which is made from a high temperature-resistant material.

53. The method according to claim 45,
wherein an instrument is used wherein the protective device is configured at a distal end of the instrument or effector.

54. The method according to claim 40, wherein an instrument is used wherein the protective device is electrically insulating or made from heat-resistant and electric arc-resistant material.

55. The method according to claim 45,
wherein an instrument is used wherein the effector comprises a sleeve or a holder for holding the electrode.

56. The method according to claim 40, wherein the protective device comprises a component which is configured spoon-shaped, finger-shaped or spatula-shaped and is pushed or pulled in a substantially straight-line movement in an axial direction of the instrument under the at least one wire and the at least one wire is threaded into the protective device (25) and is separated or distanced from the tissue.

57. The method according to claim 40, wherein the protective device comprises a screw-shaped or corkscrew shaped component that is screwed in a substantially twisting or rotating movement under the at least one wire and the at least one wire is threaded into the protective device and separated or distanced from the tissue.

58. The method according to claim 56,
wherein, by means of the component, a plurality of wires is threaded and separated or distanced from the tissue simultaneously.

59. The method according to claim 56,
wherein an instrument is used wherein the at least one wire threaded in and separated or distanced from the tissue is held at a predetermined distance from the electrode by means of the protective device thus configured.

60. The method according to claim 40, wherein the instrument is pressed, pushed or rotated such that the at least one wire slips into at least one guide that includes at least one notch which is formed on the protective device, and is fixed therein.

61. The method according to claim 60,
wherein an instrument is used, and wherein the at least one wire threaded in and/of separated or distanced from the tissue is held by means of the guide at the predetermined distance from the electrode.

62. The method according to claim 45,
wherein at least a partial region of the effector is moved in a controlled manner by means of a movement device configured on the instrument.

63. The method according to claim 62,
wherein the movement device comprises an elastically deformable device, and wherein the at least one partial region of the effector is moved via the elastically deformable device.

64. The method according to claim 62,
wherein the movement device comprises a manipulator that is a cable element or a rod element, which is connected to the effector, and wherein the at least one partial region of the effector is moved via an elastically deformable device by actuation of the manipulator.

65. The method according to wherein the at least one wire, the stent fragment or the stent is firmly held by means of a holding device configured on the protective device.

66. The method according to claim 65,
wherein the at least one wire, the stent fragment or the stent is firmly held by means of at least one barb as the holding device.

67. The method according to one claim 65,
wherein a plurality of barbs, which, for secure accommodation of the at least one wire, the stent fragment or the stent, is arranged essentially evenly spaced from one another on the protective device as the holding device, and wherein the at least one wire, the stent fragment or the stent is securely held by the plurality of barbs.

68. The method according to claim 65,
wherein the instrument is used wherein the holding device is configured on a component for threading and separating or distancing the at least one wire from the tissue.

69. The method according to claim 65,
wherein the at least one wire, the stent fragment or the stent is firmly held by means of a movable holding device for moving the at least one wired, the stent fragment or the stent.

70. The method according to claim 65,
wherein the at least one wire, the stent fragment or the stent is removed from the hollow organ by means of the holding device.

71. The method according to claim 44, wherein an instrument is used that includes a component for threading and separating or distancing the at least one wire from the tissue such that the component is moved relative to the shaft or catheter.

72. The method according to claim 71,
wherein the device component is moved in a guide device arranged on the instrument.

73. The instrument according to claim 9, wherein the fluid is argon.

74. The instrument according to claim 12, wherein the high temperature resistant material is tungsten.

75. The instrument according to claim 30, wherein the manipulator is configured as a rod element.

76. The method according to claim 49, wherein the fluid is argon.

77. The method according to claim 52, wherein the high temperature resistant material is tungsten.

78. The method according to claim 57, wherein, by means of the component, a plurality of wires is threaded and separated or distanced from the tissue simultaneously.

79. The method according to claim 57, wherein an instrument is used wherein the at least one wire threaded in and separated or distanced from the tissue is held at a predetermined distance from the electrode by means of the protective device thus configured.

80. The method according to claim 63, wherein the elastically deformable device is a bellows.
